(19) United States
(12) Patent Application Publication
(10) Pub. No.: US 2020/0121616 A1
(43) Pub. Date: Apr. 23, 2020

(54) COMPOSITIONS COMPRISING CANNABIDIOL AND HYALURONIC ACID FOR TREATING INFLAMMATORY JOINT DISEASES

(71) Applicant: BOL PHARMA LTD.

(72) Inventor: Tamir GEDO, Revadim (IL)

(21) Appl. No.: 16/304,187

(22) PCT Filed: May 24, 2017
(86) PCT No.: PCT/IL2017/050580

§ 371 Date: Nov. 23, 2018

Related U.S. Application Data
(60) Provisional application No. 62/230,576, filed on Jun. 9, 2015.

(51) Int. Cl. A61K 31/05; A61K 31/728; A61K 47/24; A61K 9/00; A61K 9/127; A61P 19/02

(52) U.S. Cl. CPC A61K 31/05; A61K 31/728; A61K 47/24; A61K 9/1277; A61K 9/127; A61P 19/02; A61K 9/0019

(57) ABSTRACT

The present invention provides compositions comprising a combination of cannabidiol (CBD) or a derivative thereof, and hyaluronic acid or a salt thereof; a phospholipid, and optionally a carrier, methods of using the compositions for treating inflammatory joint diseases, or pain or inflammation associated with such diseases, and methods for their preparation.

### FIELD OF INVENTION

This invention is directed to compositions comprising cannabidiol (CBD) or a derivative thereof and hyaluronic acid or a salt thereof, and their use in methods for treating inflammatory joint diseases. More specifically, the invention is directed to compositions comprising liposomes including CBD or a derivative thereof, suspended in hyaluronic acid or a salt thereof, and their use for treating inflammatory joint diseases.

### BACKGROUND OF THE INVENTION

### Inflammatory Joint Diseases

Joint diseases are diseases or injuries that affect human joints. Arthritis is the best-known joint disease. Diseases of the joints may be variously short-lived or exceedingly chronic, agonizingly painful or merely nagging and uncomfortable; they may be confined to one joint or may affect many parts of the skeleton.

Two principal categories are distinguished: inflammatory joint diseases in which inflammation is the principal set of signs or symptoms, and non-inflammatory joint diseases.

Arthritis is a generic term for inflammatory joint disease. Regardless of the cause, inflammation of the joints may cause pain, stiffness, swelling, and some redness of the skin about the joint. Effusion of fluid into the joint cavity is common, and examination of this fluid is often a valuable procedure for determining the nature of the disease. The inflammation may be of such a nature and of such severity as to destroy the joint cartilage and underlying bone and cause irreparable deformities. Adhesions between the articulating members are frequent in such cases, and the resulting fusion with loss of mobility is called ankylosis.

Inflammation restricted to the lining of a joint (the synovial membrane) is referred to as synovitis. Arthralgias simply are pains in the joints; as ordinarily used, the word implies that there is no other accompanying evidence of arthritis.

Bursitis, inflammation of a synovial bursa, the lubricating sac located around joints or between tendons and muscles or bones. Bursitis may be caused by infection or injury, by arthritis or gout, by calcium deposition along a tendon or joint, or by minor, usually repetitive irritation. Bursitis commonly affects the knee (“housemaid's knee”), the Achilles tendon at the back of the ankle (“soldier's heel”), the elbow (“tennis elbow”), and the bottom of the pelvis (“weaver's bottom”), but most common is bursitis of the shoulder, caused by calcium deposits and inflammation of the rotator tendon in the upper arm, spreading into the bursa above the shoulder joint. Bursitis of the shoulder may be extremely painful, making it impossible to raise the affected arm. Treatment of bursitis includes rest, heat, mild exercise, and medications that relieve inflammation and remove calcium deposits.

Joints may be infected by many types of microorganisms (bacteria, fungi, viruses) and occasionally by animal parasites. There are three routes of infection: by direct contamination, by way of the bloodstream, and by extension from adjacent bony infections (osteomyelitis). Direct contamination usually arises from penetrating wounds but may also occur during surgery on joints. Blood-borne infections may enter the joints through the synovial blood vessels. Commonly, however, foci of osteomyelitis occur first in the long bones near the end of the shaft or next to the joint. The infection then extends into the joint through natural openings or pathological breaches in the outside layer, or cortex, of the bone. Characteristically, hematogenous (blood-borne) infectious arthritis affects one joint (monarthritis) or a very few joints (oligoarthritis) rather than many of them (polyarthritis) and usually affects large joints (knee and hip) rather than small ones. Infections of the joints, like infections elsewhere in the body, often cause fever and other systemic indications of inflammation.

In several types of arthritis that resemble infectious joint disease, no causative agent has been isolated. Principal among these is rheumatoid arthritis. This disorder may appear at any age but is most usual in the fourth and fifth decades. A type that affects children is called juvenile rheumatoid arthritis. Rheumatoid arthritis typically affects the same joints on both sides of the body. Almost any movable joint can be involved, but the fingers, wrists, and knees are particularly susceptible. The joints are especially stiff when the affected person awakes. Rheumatoid arthritis is not only a disease of the joints; fatigue and anemia indicate that there is a more generalized systemic involvement. A slight fever may sometimes be present. Lesions also occur in sites outside the joints. Involvement of bursas, tendons, and tendon sheaths is an integral part of the disease. Approximately one of five affected persons has nodules in the subcutaneous tissue at the point of the elbow or elsewhere. Inflammatory changes also are found sometimes in small arteries and the pericardium—the membrane enclosing the heart.

### Cannabidiol

Cannabidiol (CBD) is one of the active cannabinoids identified in cannabis. CBD does not appear to have any psychotropic effects such as those caused by A9-tetrahydrocannabinol (THC) in marijuana, but may have effects on anxiety and anti-psychotic effect. CBD, the molecular formula of C₂₁H₃₀O₂, as depicted in formula I:

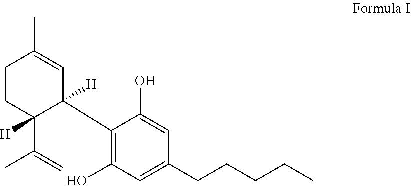 [SMILES: C=C(C)[C@@H]1CCC(C)=C[C@H]1c1c(O)cc(CCCCC)cc1O]

Specific receptors to CBD exist within synovial joints. Some studies demonstrate that the CB type 2 receptor activation pathway plays a role in the pathophysiology of osteoarthritis in mice and shows that pharmacological activation of CB2 has a protective effect. Some cannabinoids may prevent cartilage resorption, in part, by inhibiting cytokine-induced NO production by chondrocytes and also by inhibiting proteoglycan degradation. These findings appear to indicate that local elevation of CBD levels in joints might protect from osteoarthritic destruction of synovial joints.

Pharmacological studies have shown the anti-nociceptive effects of cannabinoids in different rodent models of osteoarthritis, and compelling evidence suggests an active participation of the endocannabinoid system in the pathophysiology of this disease. The ubiquitous distribution of cannabinoid receptors, together with the physiological role of the endocannabinoid system in the regulation of pain, inflammation and even joint function further support the therapeutic interest of cannabinoids for osteoarthritis.

Some evidence exists that the effect of cannabinoids might be attenuation of the inflammatory component as occurs for example in rheumatoid arthritis (RA). Increasing evidence suggests that the endocannabinoid system, especially cannabinoid receptor 2 (CB2), has an important role in the pathophysiology of RA. Many members of the endocannabinoid system are reported to inhibit synovial inflammation, hyperplasia, and cartilage destruction in RA. In particular, specific activation of CB2 may relieve RA by inhibiting not only the production of autoantibodies, proinflammatory cytokines, and matrix metalloproteinases (MMPs), but also bone erosion, immune response mediated by T cells. Indeed a synthetic cannabinoid has been shown to decrease inflammation in rheumatoid arthritis. In RA, synovial fibroblasts (SF) secrete large amounts of IL-6, IL-8 and MMPs which are crucial for cartilage destruction. RASFs are sensitive to the action of cannabinoids and they not only express cannabinoid receptors type I and II (CB1 and CB2) but also transient receptor potential channels type vanilloid (TRPV1) and ankyrin (TRPA1). The synthetic cannabinoid receptor agonist WIN55, 212-2 mesylate (WIN) demonstrated strong anti-inflammatory effects in monocytes and synovial fibroblasts only in high concentrations in a non-cannabinoid receptor dependent manner.

### Hyaluronic acid

Hyaluronic acid (HA), also called hyaluronan, is an anionic, nonsulfated glycosaminoglycan widely distributed throughout connective, epithelial, and neural tissues. It is unique among glycosaminoglycans (GAGS) in that it is nonsulfated, forms in the plasma membrane instead of the Golgi apparatus, and can be very large. It is one of the chief components of the extracellular matrix, contributing significantly to cell proliferation and migration.

Hyaluronic acid has been widely used for viscosupplementation of diseased or aged articular joints. However, recent investigations have revealed the active anti-inflammatory or chondroprotective effect of hyaluronic acid, suggesting its potential role in attenuation of joint damage (Masuko, 2009). Hyaluronan has been found to be effective in treatment of inflammatory processes in medical areas such as orthopedics, dermatology and ophthalmology, and it has been further found to be anti-inflammatory and antibacterial in gingivitis and periodontitis therapy. Due to its tissue healing properties, it could be used as an adjunct to mechanical therapy in the treatment of periodontitis (Sukumar and Drizhal, 2007)

### Treatments for Inflammatory Joint Diseases

Inflammatory joint diseases are treated with anti-inflammatory pain relievers such as non-steroidal anti-inflammatory drugs (e.g. aspirin, ibuprofen), corticosteroids (e.g. prednisone), and other medications including chemotherapy drugs, and disease-modifying anti rheumatic drugs (DMARDs, such as azathioprine, cyclosporine, methotrexate, monoclonal antibodies and pathway inhibitors). Treatments may be systemic or local, to the inflamed joint.

Systemic medications for treating inflammatory joint diseases have many side effects, including, stomach ulcers, possible increase in risk of blood clots, elevated blood fats and sugar levels, increased susceptibility to infection, etc. Local treatments also have side effects, for example, aggravation of the pain due to irritation of the joint lining by crystals in the steroid is steroid injections. Therefore, better drugs, having fewer side effects are still in demand. Additionally, it is preferable to administer a combination of drugs, which facilitates administering less of the drugs which cause more severe side effects.

### SUMMARY OF THE INVENTION

In one aspect, the present invention provides a composition comprising a combination of cannabidiol (CBD) or a derivative thereof, and hyaluronic acid or a salt thereof; a phospholipid, and optionally a carrier.

In another aspect, the present invention provides a method for treating, or reducing pain or inflammation associated with, an inflammatory joint disease, disorder or condition in a subject in need thereof, comprising administering to the subject the composition of the invention as defined above.

According to an additional aspect, the present invention provides a method for preparing the composition of the invention, wherein the composition includes cannabidiol (CBD), hyaluronic acid, phospholipid, and cholesterol, and the method comprises the following steps:
a) mixing the CBD, phospholipid and cholesterol to form a mixture;
b) sonicating and homogenizing the mixture; and
c) suspending the mixture in a solution of hyaluronic acid or a salt thereof,
   thereby forming liposomes suspended in hyaluronic acid.

According to a yet additional aspect, the present invention provides the composition of the invention as defined above, for use in treating, or reducing pain or inflammation associated with, an inflammatory joint disease, disorder or condition.

### BRIEF DESCRIPTION OF THE DRAWINGS

FIG. 1 shows the effect of a composition comprising CBD and hyaluronic acid (HA) on arthritis in a rat model. Osteoarthritis was induced in rats, which were then treated with either CBD/HA formulation (LYP, right group, n=7), or HA alone (left group, n=3). The rats were tested after a week in an incapacitance test and the percentage of weight out of their total weight that the animals were able to bear was calculated. The horizontal lines indicate the mean values. The P value was 0.0381.

### DETAILED DESCRIPTION OF THE INVENTION

The present invention is based on the finding that a combination of cannabidiol (CBD) and hyaluronic acid (HA) is more effective in ameliorating symptoms of inflammatory joint diseases compared to hyaluronic acid alone.

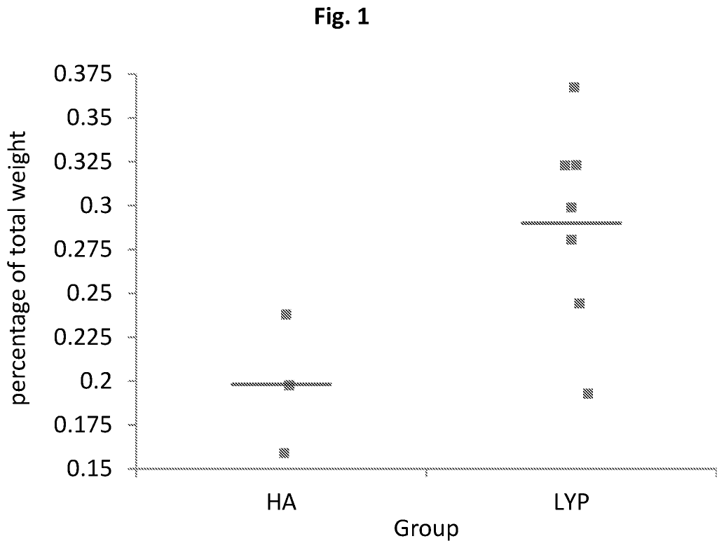

As described below in Example 2, rats induced with osteoarthritis were treated with a formulation including liposomes comprising CBD solubilized in Phospholipon® 90G (which essentially comprises phosphatidylcholines), and suspended in hyaluronic acid. As can be seen from FIG. 1, this treatment resulted in a significantly increased ability to bear pain, as compared with arthritic rats treated with hyaluronic acid alone.

Accordingly, the present invention provides a composition comprising a combination of cannabidiol (CBD) or a derivative thereof, and hyaluronic acid or a salt thereof; a phospholipid, and optionally a carrier.

In some embodiments, the composition comprises CBD. In some embodiments, the composition comprises a CBD derivative.

The term “CBD derivative” as used herein means a CBD derivative having an anti-inflammatory effect, or an analgesic effect, or having an ameliorating effect on inflammatory joint disease, disorder or conditions; or alternatively, a CBD derivative that binds to CB(1) and/or CB(2) cannabinoid receptors.

In some embodiments, a CBD derivative is selected from (−)-7-hydroxy-CBD, which is known from WO 2015/198077 to reduce triglyceride levels and treat fatty liver; (−)-CBD-7-oic acid, which is known from Haj 2015 to have an anti-inflammatory effect; and the dimethylheptyl (DMH) homolog of CBD, which is known to have an anti-inflammatory effect (Ben-Shabat 2006; Juknat 2016), and the corresponding compounds in the enantiomeric (+)-CBD series.

In some embodiments, a CBD derivative is characterized by a structure, wherein at least one of the hydroxyl substituent groups is converted to a stable form thereof. In some embodiments, a CBD derivative is cannabinol comprising a quinone ring. In one embodiment, a CBD derivative is an endocannabinoid derivative. In another embodiment, the pentyl group on the phenyl ring of the CBD is replaced with any straight-chain or branched alkyl group selected from (C₁-C₁₈)alkyl, optionally substituted.

In some embodiments the CBD is prepared from a cannabis extract. In some embodiments the term “CBD or a derivative thereof” refers to between 80% and 99% pure CBD. In some embodiments the term “CBD or a derivative thereof” refers to between 90% and 99% pure CBD. In some embodiments the term “CBD or a derivative thereof” refers to between 93% and 99% pure CBD. In some embodiments the term “CBD or a derivative thereof” refers to between 95% and 99% pure CBD. In some embodiments the term “CBD or a derivative thereof” refers to between 95% and 97% pure CBD. In some embodiments the term “CBD or a derivative thereof” refers to about 97% pure CBD. All % hereinabove are weight %.

In some embodiments, the CBD or a derivative thereof is substantially and/or essentially devoid of tetrahydrocannabinol (THC). In some embodiments, a composition of the invention, as described herein, is substantially and/or essentially devoid of THC. In one embodiment, substantially and/or essentially devoid of THC means less than 10% by weight THC. In one embodiment, substantially and/or essentially devoid of THC is less than 7% by weight THC. In one embodiment, substantially and/or essentially devoid of THC is less than 5% by weight THC. In one embodiment, substantially and/or essentially devoid of THC is less than 3% by weight THC. In one embodiment, substantially and/or essentially devoid of THC is less than 1% by weight THC. In one embodiment, substantially and/or essentially devoid of THC is less than 0.5% by weight THC. In one embodiment, substantially and/or essentially devoid of THC is less than 0.3% by weight THC. In one embodiment, substantially and/or essentially devoid of THC is less than 0.1% by weight THC.

In some embodiments, the CBD is synthetically prepared.

The term “hyaluronic acid or salt thereof” is used interchangeably with “hyaluronan”, “hyaluronic acid”, “hyaluronate”, or “HA”. The balance between the acid and the salt forms depends on the pH of the solution and the pKa of hyaluronic acid (about 2.8). Thus, at physiological pH it is mainly in a salt form.

The molecular weight of hyaluronan can be anywhere from several kilo dalton (kDa) to over 10⁷ dalton. As previously shown, high molecular weight hyaluronan is immunosuppressive, antiangiogenic and anti-inflammatory, and was shown to protect against lymphocyte-mediated cytolysis (McBride and Bard, 1979), suppress septic responses to lipopolysaccharides, maintain immune tolerance, induce production of immunosuppressive macrophages, and reduce expression of inflammatory cytokines. Such hyaluronan was further found to have antiaging and anticancer effects; and are known to cause cell cycle arrest, mediated by transmembrane association between cluster of differentiation 44 (CD44) and the intracellular protein merlin, and to protect against apoptosis by a mechanism mediated by nuclear factor kappa-B (NF-κB).

Accordingly, in some embodiments, the molecular weight of the hyaluronic acid is above about 5×10⁵ dalton, e.g. between about 5×10⁵ dalton and about 10⁷dalton, between about 5'10⁵ dalton and about 8×10⁵ dalton, between about 5'10³ dalton and about 1×10⁶ dalton, between about 8×10⁵ dalton and about 2×10⁶ dalton, between about 1×10⁶ dalton and about 7×10⁶ dalton, between about 2×10⁶ dalton and about 6×10⁶ dalton, between 3×10⁶ dalton and 6×10⁶ dalton, and between about 5×10⁶ dalton and about 10⁷ dalton. In particular the molecular weight of the hyaluronic acid is about 6×10⁶ dalton.

In some embodiments, the hyaluronic acid is a high molecular weight hyaluronic acid, i.e. of a molecular weight above about 500,000 dalton. In some embodiments, the hyaluronic acid is an intermediate molecular weight hyaluronic acid, i.e. of a molecular weight of about 60,000-500,000 dalton.

In some embodiments, the hyaluronic acid is crosslinked. Crosslinking may be carried out by any acceptable method, such as attaching thiol groups, methacrylates, hexadecylamides, or tyramine groups; or directly with formaldehyde (Hylan-A), or with divinylsulfone (trade name: Hylan-B). Hyaluronic acid can be cross-linked via various functional groups, e.g., via the acetyl group (NHCOCH3) after deacetylation, via the carboxylic acid group, or via one of the hydroxyl groups. Hyaluronic acid can be cross-linked with glutaraldehyde via hemiacetal formation.

The hyaluronic acid salt may be any suitable cationic salt of hyaluronic acid. In some embodiments, the hyaluronic acid salt is a pharmaceutically acceptable salt. Examples of hyaluronan salts include, without limiting, alkaline metal or alkaline earth metal salts of hyaluronan, salts, such as lithium, sodium, potassium, cesium, calcium and magnesium salt; and in particular hyaluronan sodium salt and hyaluronan potassium salt.

Accordingly, in some embodiments, the salt of hyaluronic acid is sodium or potassium hyaluronate.

The hyaluronic acid may be from an animal source, or the hyaluronic acid may be prepared by microbiology methods in bacteria.

The phospholipid for use in the present invention is any phospholipid which can be used to bring the CBD into suspension in order to enable preparation of a suspension with the hyaluronic acid. In some embodiments, the phospholipid for use in the present invention is any phospholipid which can be used to bring the CBD into suspension in order to enable preparation of an injectable suspension with the hyaluronic acid.

The term “phospholipid” as used herein refers to any phospholipid, a mixture of phospholipids, a product or a mixture which essentially comprises phospholipids, such as lecithin or lecithin-based products, or lecithin-like substances.

According to certain embodiments, the phospholipid comprises a phosphatidylcholine, a hydrogenated phosphatidylcholine, a lysophosphatidylcholine, dipalmitoylphosphatidylcholine, distearoylphosphatidylcholine, dimyristoylphosphatidylcholine, dilauroylphosphatidylcholine, dioleylphosphatidylcholine, 1-palmitoyl 2-oleyl phosphatidylcholine, a glycerophospholipid, sphingomyelin, cardiolipin, a phosphatidylserine, a phosphatidylglycerol, a phosphatidylinositol, a phosphatidic acid, a phosphatidylcholine-based product, a glycolipid, a plasmalogen, a phosphosphingolipid, Asolectin, lecithin, or a lecithin-like substance, or a mixture thereof.

According to certain embodiments, the phospholipid is from a natural source. According to certain embodiments, the natural source is an animal source. According to certain embodiments, the natural source is a plant source. According to some embodiments, the phospholipid is from egg or from soybean. According to certain embodiments, the phospholipid is from a synthetic source.

According to certain embodiments, the lecithin is egg lecithin or soybean lecithin; the lecithin-like substance is lecithin egg yolk or soybean oil; or the phosphatidylcholine-based product is Phospholipon® 50, Phospholipon® 75, Phospholipon® 85G, Phospholipon® 90G, Phospholipon® 80H, Phospholipon® 90H, Phospholipon® E25, Phospholipon® E35, Phospholipon® E, Phospholipon® LPC2O, Phospholipon® LPC25, or Phospholipon® LPC65.

Phospholipon® 50, Phospholipon® 75, Phospholipon® 85G and. Phospholipon® 90G, essentially consist of soybean lecithin and phospholipids; Phospholipon® 80H and Phospholipon® 90H, essentially consist of hydrogenated soybean lecithin and phospholipids; Phospholipon® E25, Phospholipon® E35 and Phospholipon® E, essentially consist of egg yolk lecithin and phospholipids; and Phospholipon® LPC2O, Phospholipon® LPC25 and Phospholipon® LPC65, essentially consist of partially hydrolyzed soybean lecithin. Phospholipon® 90G comprises at least 90% phosphatidylcholine.

Phosphatidylcholines are a class of phospholipids that incorporate choline as a headgroup. They are a major component of biological membranes and can be easily obtained from a variety of readily available sources, such as egg yolk or soybeans, from which they are mechanically or chemically extracted using hexane. They are also a member of the lecithin group of yellow-brownish fatty substances occurring in animal and plant tissues. Dipalmitoyl phosphatidylcholine is a major component of pulmonary surfactant. Phosphatidylcholines are such a major component of lecithin that in some contexts the terms are sometimes used as synonyms. However, lecithin extracts consist of a mixture of phosphatidylcholine and other compound. According to some embodiments, the phosphatidylcholine is from egg or from soybean.

In some embodiments, the phospholipid comprises a phosphatidylcholine or a phosphatidylcholine-based product.

In some embodiments, the phosphatidylcholine-based product is Phospholipon® 90G.

The phospholipid or phosphatidylcholine described above may form liposomes which embed or non-covalently bind the CBD. Cholesterol may be added to liposomes to increase stability.

Accordingly, in some embodiments, the composition further comprises cholesterol.

In some embodiments, the composition of the invention comprises a combination of CBD, and high molecular weight hyaluronic acid or a salt thereof, and any phospholipid as defined in any of the above embodiments. In some embodiments, the composition essentially consists of CBD, high molecular weight hyaluronic acid or a salt thereof, and phospholipid. In some embodiments, the composition essentially consists of CBD, high molecular weight hyaluronic acid or a salt thereof, cholesterol, and a phospholipid. In some embodiments, the composition of the invention comprises a combination of CBD and high molecular weight hyaluronic acid or a salt thereof, wherein the hyaluronic acid or salt thereof is cross-linked.

According to some embodiments, the composition of the invention comprises a combination of CBD and high molecular weight hyaluronic acid or a salt thereof, and a phospholipid comprising, or which is selected from, a phosphatidylcholine, a hydrogenated phosphatidylcholine, a lysophosphatidylcholine, dipalmitoylphosphatidylcholine, distearoylphosphatidylcholine, dimyristoylphosphatidylcholine, dilauroylphosphatidylcholine, dioleylphosphatidylcholine, 1-palmitoyl 2-oleyl phosphatidylcholine, a glycerophospholipid, sphingomyelin, cardiolipin, a phosphatidylserine, a phosphatidylglycerol, a phosphatidylinositol, a phosphatidic acid, a phosphatidylcholine-based product, a glycolipid, a plasmalogen, a phosphosphingolipid, Asolectin, lecithin, or a lecithin-like substance, or a mixture thereof.

According to certain embodiments, the composition of the invention comprises a combination of CBD and high molecular weight hyaluronic acid or a salt thereof, and lecithin that is egg lecithin or soybean lecithin; lecithin-like substance that is lecithin egg yolk or soybean oil; or phosphatidylcholine-based product that is Phospholipon® 50, Phospholipon® 75, Phospholipon® 85G, Phospholipon® 90G, Phospholipon® 80H, Phospholipon® 90H, Phospholipon® E25, Phospholipon® E35, Phospholipon® E, Phospholipon® LPC20, Phospholipon® LPC25, or Phospholipon® LPC65.

According to certain embodiments, the composition of the invention comprises a combination of CBD and high molecular weight hyaluronic acid or a salt thereof, and phosphatidylcholine-based product comprising Phospholipon® 50, Phospholipon® 75, Phospholipon® 85G or Phospholipon® 90G.

According to certain embodiments, the composition of the invention comprises a combination of CBD and high molecular weight hyaluronic acid or a salt thereof, and a phospholipid comprising, or selected from, a phosphatidylcholine, or a phosphatidylcholine-based product.

According to certain embodiments, the composition of the invention comprises a combination of CBD and high molecular weight hyaluronic acid or a salt thereof, and a phosphatidylcholine-based product comprising Phospholipon® 90G.

In some embodiments, the composition of the invention essentially consists of CBD, high molecular weight hyaluronic acid or a salt thereof, and a phosphatidylcholine or Phospholipon® 90G. In some embodiments, the composition of the invention essentially consists of CBD, high molecular weight hyaluronic acid or a salt thereof, cholesterol, and a phosphatidylcholine or Phospholipon® 90G.

The hyaluronic acid of any of the above embodiments may be crosslinked as discussed above. The hyaluronic acid of any of the above embodiments may be in the form of sodium or potassium salt.

The term “essentially consisting of” means that the composition may further comprise non-active ingredients such as solvents.

In some embodiments, the concentration of CBD in the formulation is between 3% W/V and 7% W/V. In some embodiments, the concentration of CBD in the formulation is between 4% W/V and 6% W/V. In some embodiments, the concentration of CBD in the formulation is about 5% W/V. In some embodiments, the concentration of CBD in the formulation is between 10 and 100 mg/ml. In some embodiments, the concentration of CBD in the formulation is between 30 and 70 mg/ml. In some embodiments, the concentration of CBD in the formulation is about 50 mg/ml.

In some embodiments, the concentration of the hyaluronic acid in the formulation is between 0.1% W/V and 0.5% W/V. In some embodiments, the concentration of the hyaluronic acid in the formulation is 0.1%, 0.2%, 0.3%, 0.4% or 0.5%, and in particular about 0.24% W/V.

In some embodiments, the concentration of the hyaluronic acid in the formulation is between 1 mg/ml and 5 mg/ml. In some embodiments, the concentration of the hyaluronic acid in the formulation is about 2.4 mg/ml.

In some embodiments, the concentration of phospholipid in the formulation is between 10% W/V and 20% W/V. In some embodiments, the concentration of phospholipid in the formulation is about 10%, 11%, 12%, 13%, 14% 15%, 16%, 17%, 18%, 19% or 20%, and in particular 16% W/V. In some embodiments, the concentration of phospholipid in the formulation is between 100 mg/ml and 200 mg/ml. In some embodiments, the concentration of phospholipid in the formulation is about 160 mg/ml.

In some embodiments, the weight ratio of CBD/phospholipid is higher than 1/10. In some embodiments, the weight ratio of CBD/phospholipid is between 1/10 and 1/2. In some embodiments, the weight ratio of CBD phospholipid is between ¹/₅ and 1/2. In some embodiments, the weight ratio of CBD/phospholipid is about 1/3.

In some embodiments, the composition comprises liposomes formed by the phospholipid as described in the embodiments above, and the CBD or derivative thereof is non-covalently attached to the liposomes. In some embodiments, the CBD is embedded in the bi-layer membrane of the liposomes.

In some embodiments, the liposomes are suspended in hyaluronic acid. The hyaluronic acid is the same as the hyaluronic acid or salt thereof as described in any of the embodiments above. In some embodiments, at least some of the hyaluronic acid is inside the liposome. In some embodiments, the hyaluronic acid is crosslinked. In some embodiments, the hyaluronic acid is sodium hyaluronate or potassium hyaluronate.

In some embodiments, the liposomes further comprise cholesterol. In some embodiments, the concentration of cholesterol in the composition of the invention is between 20 and 50 mg/ml. In some embodiments, the concentration of the cholesterol is about 40 mg/ml.

In sonic embodiments, the composition of the invention comprises CBD; phosphatidylcholine, or Phospholipon® 90G; high molecular weight, crosslinked, sodium hyaluronate; and cholesterol, wherein liposomes are formed from the phosphatidylcholine or Phospholipon® 90G, and the cholesterol; the CBD is non-covalently attached to the liposomes; and the liposomes are suspended in the high molecular weight, crosslinked sodium hyaluronate.

In some embodiments, a composition as described herein is in the form of a liquid. In some embodiments, a composition as described herein is in the form of a gel. In some embodiments, the composition comprises a buffer. In some embodiments, the composition comprises a buffer keeping the pH of the solution at a physiological pH.

In some embodiments, the composition further includes a co-solvent. In some embodiments, a co-solvent is a mixture of miscible solvents for solubilizing water-insoluble ingredients of the invention. In sonic embodiments, a co-solvent is composed of one organic solvent and water. In some embodiments, a co-solvent comprises: propylene glycol, PEG 400, ethanol, water, a surfactant, glycerin, propylene glycol, ethanol, polyethylene glycol 300, polyethylene glycol 400, dimethylacetamide (DMA), N-methyl-2-pyrrolidone (NMP; Pharmasolve), dimethylsulfoxide (DMSO), Solutol HS 15, Cremophor EL, Cremophor RH 60, and polysorbate 80. In some embodiments, a co-solvent comprises a compound described in:

Robert G. Strickley: Solubilizing Excipients in Oral and Injectable Formulations. Pharmaceutical Research, Vol. 21, No. 2 pp. 201-230, February 2004, which is hereby incorporated by reference in its entirety.

In some embodiments, a composition as described herein further comprises cannabichromene (CBC), cannabigerol (CBG), cannabinol (CBN), or any combination thereof. In one embodiment, a composition as described herein further comprises at least two compounds selected from the group comprising: cannabichromene (CBC), cannabigerol (CBG), cannabinol (CBN), or any combination thereof.

In some embodiments, a composition as described herein further comprises an oil soluble vitamin. In one embodiment, an oil soluble vitamin is vitamin E. In one embodiment, an oil soluble vitamin is vitamin D. In some embodiments, an oil soluble vitamin is vitamin K. In some embodiments, an oil soluble vitamin is vitamin A. In some embodiments, an oil soluble vitamin is any combination of vitamin E, vitamin D, vitamin K and vitamin A.

In some embodiments, the composition as described herein further comprises an additional glycosaimnoglycan (“GAG”). In some embodiments, the composition as described herein further comprises chondroitin sulfate (“CSA”). In some embodiments, a composition as described herein comprises a polyol. In sonic embodiments, a composition as described herein further comprises at least one additional anti-inflammatory agent polyol.

In some embodiments, the carrier is a pharmaceutically acceptable carrier. In some embodiments the composition of the invention is a pharmaceutical composition, wherein the hyaluronic acid or salt thereof is a pharmaceutically acceptable salt of hyaluronic acid as defined above, and the composition comprises a pharmaceutically acceptable carrier.

In some embodiments, the described compositions further comprise at least one pharmaceutically acceptable carrier, diluent, excipient and/or additive.

Cannabidiol is insoluble in water but soluble in organic solvents, such as oil. Accordingly, CBD can be formulated for use in the described methods through use of any organic solvent known to the pharmaceutical arts, including, but not limited to edible oils. When formulated for oral administration, any edible oil can be used in the CBD formulation, including olive oil.

In some embodiments, a composition as described herein is formulated to a suitable route of administration, such as: oral, rectal, transmucosal, transnasal, intestinal or parenteral delivery, including intramuscular, subcutaneous and intramedullary injections as well as intrathecal, direct intraventricular, intravenous, intraperitoneal, intranasal, or intraocular injections.

in some embodiments, the pharmaceutical compositions are administered by intravenous, intra-arterial, or intramuscular injection of a liquid preparation. In some embodiments, liquid formulations include solutions, suspensions, dispersions, emulsions, oils and the like. In one embodiment, the pharmaceutical compositions are administered intravenously, and are thus formulated in a form suitable for intravenous administration. In another embodiment, the pharmaceutical compositions are administered intra-arterially, and are thus formulated in a form suitable for intra-arterial administration. In another embodiment, the pharmaceutical compositions are administered intramuscularly, and are thus formulated in a form suitable for intramuscular administration.

In some embodiments, injectables of the invention are formulated in aqueous solutions. In one embodiment, injectables of the invention are formulated in physiologically compatible buffers such as Hank's solution, Ringer's solution, or physiological salt buffer. In some embodiments, for transmucosal administration, penetrants appropriate to the barrier to be permeated are used in the formulation. Such penetrants are generally known in the art.

In some embodiments, the preparations described herein are formulated for parenteral administration, e.g., by bolus injection or continuous infusion. In some embodiments, formulations for injection are presented in unit dosage form, e.g., in ampoules or in multidose containers with optionally, an added preservative. In some embodiments, compositions are suspensions, solutions or emulsions in oily or aqueous vehicles, and contain formulatory agents such as suspending, stabilizing and/or dispersing agents.

In some embodiments, pharmaceutical compositions for parenteral administration include aqueous solutions of the active preparation in water-soluble form. Additionally, suspensions of the active ingredients, in some embodiments, are prepared as appropriate oily or water based injection suspensions. Suitable lipophilic solvents or vehicles include, in some embodiments, fatty oils such as sesame oil, or synthetic fatty acid esters such as ethyl oleate, triglycerides or liposomes. Aqueous injection suspensions contain, in some embodiments, substances, which increase the viscosity of the suspension, such as sodium carboxymethyl cellulose, sorbitol or dextran. In another embodiment, the suspension also contain suitable stabilizers or agents which increase the solubility of the active ingredients to allow for the preparation of highly concentrated solutions.

In some embodiments, a composition as described herein is an intra-articular injectable composition. In some embodiments, a composition as described herein is a viscosupplementation composition. In some embodiments, a composition as described herein is in a gel form or semi-gel form. In some embodiments, a dosage form of a composition comprises or consists of 0.5 to 5 ml of a composition as described herein.

In some embodiments, a dosage form of a composition comprises or consists of 0.5 to 2 nil of a composition as described herein. In some embodiments, a dosage form of a composition comprises or consists of 1 to 3 ml of a composition as described herein. In some embodiments, a dosage form of a composition as described herein is injected 1 to 5 times a week. In some embodiments, a dosage form of a composition as described herein is injected 2 to 5 times a week. In some embodiments, a dosage form of a composition as described herein is injected for a duration of one week to a year. In some embodiments, a dosage form of a composition as described herein is injected for a duration of one month to a year. In some embodiments, a dosage form of a composition as described herein is injected for a duration of two months, or a week to ten months.

The pharmaceutically-acceptable carriers suitable for the preparation of unit dosage forms of a composition as described herein for peroral administration are well-known in the art. In some embodiments, tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. In one embodiment, glidants such as silicon dioxide can be used to improve flow characteristics of the powder-mixture.

In one embodiment, coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents. In some embodiments, the selection of carrier components depends on secondary considerations like taste, cost, and shelf stability, which are not critical for the purposes of this invention, and can be readily made by a person skilled in the art.

In some embodiments, the oral dosage form comprises predefined release profile. In one embodiment, the oral dosage form of the present invention comprises a dosage form composition) or dosage forms having different release profile for hyaluronic acid and for CBD. In some embodiments, the oral dosage form of the present invention comprises a dosage form composition) or dosage forms having the same release profile for hyaluronic acid and for CBD. In some embodiments, the oral dosage form of the present invention comprises an extended release tablets, capsules, lozenges or chewable tablets. In some embodiments, the oral dosage form of the present invention comprises a slow release tablets, capsules, lozenges or chewable tablets. In some embodiments, the oral dosage form of the present invention comprises an immediate release tablets, capsules, lozenges or chewable tablets. In some embodiments, the oral dosage form is formulated according to the desired release profile of the pharmaceutical active ingredient as known to one skilled in the art.

Peroral compositions, in some embodiments, comprise liquid solutions, emulsions, suspensions, and the like. In some embodiments, pharmaceutically-acceptable carriers suitable for preparation of such compositions are well known in the art.

In some embodiments, compositions for use in the methods of this invention comprise solutions or emulsions, which in some embodiments are aqueous solutions or emulsions comprising a safe and effective amount of the compounds of the present invention and optionally, other compounds, intended for topical intranasal administration.

Further, in some embodiments, the pharmaceutical compositions are administered topically to body surfaces, and are thus formulated in a form suitable for topical administration. Suitable topical formulations include gels, ointments, creams, lotions, drops and the like. For topical administration, the compounds of the present invention are combined with an additional appropriate therapeutic agent or agents, prepared and applied as solutions, suspensions, or emulsions in a physiologically acceptable diluent with or without a pharmaceutical carrier.

In one embodiment, pharmaceutical compositions of the present invention are manufactured by processes well known in the art, e.g., by means of conventional mixing, dissolving, granulating, dragee-making, levigating, emulsifying, encapsulating, entrapping or lyophilizing processes.

In some embodiments, pharmaceutical compositions for use in accordance with the present invention is formulated in conventional manner using one or more physiologically acceptable carriers comprising excipients and auxiliaries, which facilitate processing of the active ingredients into preparations which, can be used pharmaceutically. In one embodiment, formulation is dependent upon the route of administration chosen.

The compositions also comprise, in some embodiments, preservatives, such as benzalkonium chloride and thimerosal and the like; chelating agents, such as edetate sodium and others; buffers such as phosphate, citrate and acetate; tonicity agents such as sodium chloride, potassium chloride, glycerin, mannitol and others; antioxidants such as ascorbic acid, acetylcystine, sodium metabisulfote and others; aromatic agents; viscosity adjustors, such as polymers, including cellulose and derivatives thereof; and polyvinyl alcohol and acid and bases to adjust the pH of these aqueous compositions as needed. The compositions also comprise, in some embodiments, local anesthetics or other actives. The compositions can be used as sprays, mists, drops, and the like.

In some embodiments, the pharmaceutical composition or compositions are delivered in a controlled release system formulated for intravenous infusion, implantable osmotic pump, transdermal patch, liposomes, intra-articular, or other modes of administration. In one embodiment, a pump is used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In yet another embodiment, a controlled release system can be placed in proximity to the therapeutic target, i.e., the brain, thus requiring only a fraction of the systemic dose (see, e.g., Goodson, in Medical Applications of Controlled Release, supra, vol. 2, pp. 115-138 (1984). Other controlled release systems are discussed in the review by Langer (Science 249:1527-1533 (1990).

In some embodiments, the active ingredient is in powder form for constitution with a suitable vehicle, e.g., sterile, pyrogen-free water based solution, before use. Compositions are formulated, in some embodiments, for atomization and inhalation administration. In another embodiment, compositions are contained in a container with attached atomizing means.

In one embodiment, the preparation of the present invention is formulated in rectal compositions such as suppositories or retention enemas, using, e.g., conventional suppository bases such as cocoa butter or other glycerides.

In some embodiments, pharmaceutical compositions suitable for use in context of the present invention include compositions wherein the active ingredients are contained in an amount effective to achieve the intended purpose. In some embodiments, a therapeutically effective amount means an amount of active ingredients effective to prevent, alleviate or ameliorate symptoms of disease or prolong the survival of the subject being treated.

Some examples of substances which can serve as pharmaceutically-acceptable carriers or components thereof are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearin acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the Tween™ brand emulsifiers; wetting agents, such sodium lauryl sulfate; coloring agents; flavoring agents; tableting agents, stabilizers; antioxidants; preservatives; pyrogen-free water; isotonic saline; and phosphate buffer solutions. The choice of a pharmaceutically-acceptable carrier to be used in conjunction with the compound is basically determined by the way the compound is to be administered. If the subject compound is to be injected, in one embodiment, the pharmaceutically-acceptable carrier is sterile, physiological saline, with a blood-compatible suspending agent, the pH of Which has been adjusted to about 7.4.

In addition, the compositions further comprise binders (e.g. acacia, cornstarch, gelatin, carbomer, ethyl cellulose, guar gum, hydroxypropyl cellulose, hydroxypropyl methyl cellulose, povidone), disintegrating agents (e.g. cornstarch, potato starch, alginic acid, silicon dioxide, croscarmelose sodium, crospovidone, guar gum, sodium starch glycolate), buffers (e.g., Tris-HCl,, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., glycerol, polyethylene glycerol), anti-oxidants (e.g., ascorbic acid, sodium metabisulfite, butylated hydroxyanisole), stabilizers (e.g. hydroxypropyl cellulose, hyroxypropylmethyl cellulose), viscosity increasing agents (e.g. carbomer, colloidal silicon dioxide, ethyl cellulose, guar gum), sweeteners (e.g. aspartame, citric acid), preservatives (e.g., Thimerosal, benzyl alcohol, parabens), lubricants (e.g. stearic acid, magnesium stearate, polyethylene glycol, sodium lauryl sulfate), flow-aids (e.g. colloidal silicon dioxide), plasticizers (e.g. diethyl phthalate, triethyl citrate), emulsifiers (e.g. carbomer, hydroxypropyl cellulose, sodium lauryl sulfate), polymer coatings (e.g., poloxamers or poloxamines), coating and film forming agents (e.g. ethyl cellulose, acrylates, polymethacrylates) and/or adjuvants.

Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, cellulose (e.g. Avicel™, RC-591), tragacanth and sodium alginate; typical wetting agents include lecithin and polyethylene oxide sorbitan (e.g. polysorbate 80). Typical preservatives include methyl paraben and sodium benzoate. In another embodiment, peroral liquid compositions also contain one or more components such as sweeteners, flavoring agents and colorants disclosed above.

Also comprehended by the invention are particulate compositions coated with polymers (e.g. poloxamers or poloxamines) and the compound coupled to antibodies directed against tissue-specific receptors, ligands or antigens or coupled to ligands of tissue-specific receptors.

In some embodiments, preparation of effective amount or dose can be estimated initially from in vitro assays. In one embodiment, a dose can be formulated in animal models and such information can be used to more accurately determine useful doses in humans.

In some embodiments, toxicity and therapeutic efficacy of the active ingredients described herein can be determined by standard pharmaceutical procedures in vitro, in cell cultures or experimental animals. In one embodiment, the data obtained from these in vitro and cell culture assays and animal studies can be used in formulating a range of dosage for use in human. In one embodiment, the dosages vary depending upon the dosage form employed and the route of administration utilized. In one embodiment, the exact formulation, route of administration and dosage can be chosen by the individual physician in view of the patient's condition. [See e.g., Fingl, et al., (1975) “The Pharmacological Basis of Therapeutics”, Ch. 1 p.1].

In another aspect, the present invention provides a method for treating an inflammatory joint disease, disorder or condition, or for alleviating or reducing pain or inflammation associated with the inflammatory joint disease, disorder, or condition in a subject in need thereof, comprising administering to the subject the composition of the invention according to any one of the embodiments defined above.

In some embodiments, the composition being administered according to the invention comprises a combination of cannabidiol (CBD) or a derivative thereof, and hyaluronic acid or a salt thereof; a phospholipid, and optionally a carrier.

in some embodiments, the composition being administered according to the invention comprises CBD. In some embodiments, the composition being administered according to the invention comprises a high molecular weight hyaluronic acid.

In some embodiments, the composition being administered according to the invention comprises a phosphatidylcholine or a phosphatidylcholine-based product.

In some embodiments, the composition being administered according to the invention comprises Phospholipon® 90G.

In some embodiments, the composition being administered according to the invention further comprises cholesterol.

in some embodiments, the composition being administered according to the invention comprises CBD and a high molecular weight hyaluronic acid.

In some embodiments, the composition being administered according to the invention comprises CBD, a high molecular weight hyaluronic acid, and a phosphatidylcholine or a phosphatidylcholine-based product.

In some embodiments, the composition being administered according to the invention comprises CBD, a high molecular weight hyaluronic acid, and a Phospholipon® 90G.

In some embodiments, the composition being administered according to the invention.

comprises liposomes formed by the phospholipid, and optionally cholesterol, if present, and wherein the CBD or derivative thereof is non-covalently attached to the liposomes.

In some embodiments, the composition being administered according to the invention comprises CBD; phosphatidylcholine or Phospholipon® 90G; high molecular weight, crosslinked, sodium hyaluronate; and cholesterol, wherein liposomes are formed from the phosphatidylcholine or Phospholipon® 90G, and the cholesterol; the CBD is non-covalently attached to the liposomes; and the liposomes are suspended in the high molecular weight, crosslinked sodium hyaluronate.

In one embodiment, treating an inflammatory joint disease comprises ameliorating or inhibiting symptoms associated with the inflammatory joint disease, disorder, or condition. such as pain, inflammation, impairment in joint movement, cartilage degradation, subchondral bone sclerosis, osteophyte formation, or any combination thereof. In one embodiment, treating an inflammatory joint disease comprises inhibiting disease progression.

In some embodiments, the disease, disorder or condition is selected from osteoarthritis, amyloidosis, arthritis bursitis, diffuse idiopathic skeletal hyperostosis (DISH), a ganglion cyst, gout, ankylosing spondylitis, lumbar spinal stenosis, hydroxyapatite juvenile arthritis, pseudogout, SAPHO syndrome, rheumatoid arthritis, reactive arthritis, psoriatic arthritis, sacroiliac joint pain, septic arthritis, Still's disease, and synovitis.

In some embodiments, the disease, disorder or condition is osteoarthritis.

In some embodiments, the administration is by injection.

In one embodiment, administering is by an intra-articular injection into an inflamed joint. In some embodiments, injecting into an inflamed joint is joint injection. In some embodiments, the methods described herein include joint aspiration. In some embodiments, the methods described herein include joint aspiration prior to injecting a composition of the invention into an inflamed joint. In some embodiments, administering is injecting into an inflamed soft tissue next to a joint (such as bursa). In some embodiments, administering is injecting into a soft tissue next to an inflamed joint.

In some embodiments, administering is by topical administration. In some embodiments, administering is by oral administration or by systemic administration, such as by injection. Oral administration of a composition as described herein, in one embodiment, comprises a unit dosage form comprising tablets, capsules, lozenges, chewable tablets, suspensions, emulsions and the like. Such unit dosage forms comprise a safe and effective amount of the desired compound, or compounds.

In some embodiments, depending on the severity and responsiveness of the condition to be treated, dosing can be of a single or a plurality of administrations, with course of treatment lasting from several days to several weeks or until cure is effected or diminution of the disease state is achieved.

In some embodiments, the amount of a composition to be administered will, of course, be dependent on the subject being treated, the severity of the affliction, the manner of administration, the judgment of the prescribing physician, etc.

In some embodiments, compositions including the preparation of the present invention formulated in a compatible pharmaceutical carrier are also be prepared, placed in an appropriate container, and labeled for treatment of an indicated condition.

In some embodiments, compositions of the present invention are presented in a pack or dispenser device, such as an FDA approved kit, which contain one or more unit dosage forms containing the active ingredient. In some embodiments, the pack, for example, comprise metal or plastic foil, such as a blister pack. In some embodiments, the pack or dispenser device is accompanied by instructions for administration. In some embodiments, the pack or dispenser is accommodated by a notice associated with the container in a form prescribed by a governmental agency regulating the manufacture, use or sale of pharmaceuticals, which notice is reflective of approval by the agency of the form of the compositions or human or veterinary administration. Such notice, in some embodiments, is labeling approved by the U.S. Food and Drug Administration for prescription drugs or of an approved product insert.

In some embodiments, compositions of the present invention comprise a volatile oil. In some embodiments, compositions of the present invention comprise a volatile oil obtained from turmeric. Ar-turmerone, in some embodiments, is a constituent of a volatile oil. In some embodiments, compositions of the present invention comprise a water suspension to which oil is added for forming a mixture.

In some embodiments, the composition or compositions exert their inflammation inhibitory activity only in sites of inflammation. In some embodiments, inhibiting inflammation is specifically targeting inflammatory sites. In another embodiment, inhibiting inflammation is inhibiting an inflammation mediator at a site of inflammation and riot at a site of no inflammatory activity.

In some embodiments, the CBD or derivative thereof is being slowly released from the liposome following administration.

In another aspect, the invention provides a method of preparing the composition of the invention, comprising the steps of:
a) mixing CBD or a derivative thereof, phospholipid, and cholesterol to form a mixture;
b) sonicating and homogenizing the mixture; and
c) suspending the mixture in a solution of hyaluronic acid or a salt thereof;
thereby forming liposomes suspended in hyaluronic acid.

In some embodiments CBD or a derivative thereof in a) is CBD. In some embodiments CBD or a derivative thereof in a) is a derivative of CBD.

In some embodiments, the mixture in a) is suspended in a suitable organic volatile solvent such as ethanol, which is vaporized following sonicating and homogenizing. In some embodiments, after vaporizing the organic volatile solvent, the pellet is suspended in an aqueous solvent, preferably a solvent having physiological or near-physiological pH, such as PBS. In some embodiments, the aqueous solvent is the solution of c) optionally comprising a salt of hyaluronic acid, optionally crosslinked. In some embodiments, following vaporizing the organic volatile solvent, the pellet is resuspended in an aqueous solution suitable for injection into a human body.

In some embodiments, the CBD is more than 90% pure. In some embodiments, the CBD is between 90% and 99% pure. In some embodiments, the CBD is between 95% and 99% pure. In some embodiments, the CBD is about 97% pure. The % is by weight.

In some embodiments, the CBD or a derivative thereof is substantially and/or essentially devoid of tetrahydrocannabinol (THC), as defined in embodiments above. In some embodiments, a composition as described herein is substantially and/or essentially devoid of THC, as defined in embodiments above.

In some embodiments, the weight ratio of CBD/phospholipid is higher than 1/10. In some embodiments, the weight ratio of CBD/phospholipid is between 1/10 and 1/2. In some embodiments, the weight ratio of CBD/phospholipid is between 1/5 and 1/2. In some embodiments, the weight ratio of CBD/phospholipid is about 1/3.

In some embodiments, the composition as described herein has a synergistic effect between CBD and hyaluronic acid, such as a synergistic anti-inflammatory effect or synergistic effect in reducing the severity of pain associated with the inflammatory disease disorder or condition.

In some embodiments, administering the compositions of the invention enables using lower doses of presently used drugs for treating inflammatory joint diseases, disorders or conditions, thereby reducing their side-effects. Non-limiting examples of medications used for treating inflammatory joint diseases are hyaluronic acid, non-steroidal anti-inflammatory drugs (e.g. aspirin, ibuprofen), corticosteroids (e.g. prednisone), chemotherapy drugs, disease-modifying anti rheumatic drugs (DMARDs, e.g. azathioprine, cyclosporine, methotrexate, monoclonal antibodies and specific pathway inhibitors). In some embodiments, administration of the composition of the invention for treating inflammatory joint diseases, disorders or conditions facilitates using sub-therapeutic doses of the presently used drugs, or even eliminates the need for the presently used drugs altogether.

In some embodiments, the compositions of the invention are administered in combination with medications used for treating inflammatory joint diseases, such as non-steroidal anti-inflammatory drugs (e.g. aspirin, ibuprofen), corticosteroids (e.g. prednisone), chemotherapy drugs, disease-modifying anti rheumatic drugs (DMARDs, e.g. azathioprine, cyclosporine, methotrexate, monoclonal antibodies and specific pathway inhibitors).

In another aspect, the invention provides a composition according to any of the embodiments described above, for treating an inflammatory disease disorder or condition, or for reducing the severity of inflammation or pain associated with the inflammatory disease disorder or condition.

In some embodiments, the composition if formulated for injection.

In some embodiments, the composition is formulated for intra-articular injection.

in some embodiments, the composition as described herein is used to inhibit inflammation. In some embodiments, the composition as described herein is used to alleviate joint pain. In some embodiments, the composition as described herein is further used to rebuild a connective tissue.

The disease, disorder, or conditions mentioned here is the same as listed above, with reference to methods of treatment.

The term “about”, as used herein means that values of 10% or less above or below the indicated values are also included.

The term “treating” or “treatment” as used herein as used herein refers to means of obtaining a desired physiological effect. The effect may be therapeutic in terms of partially or completely curing a disease and/or symptoms attributed to the disease. The term refers to inhibiting the disease, i.e. arresting its development; or ameliorating the disease, i.e. causing regression of the disease.

### EXAMPLES

### Materials

Phosphatidylcholine was purchased from Lipoid AG (Phospholipon® 90G (PL9OG), Cat No. 368202, including more than 96% m/m phsphatidylcholine), cholesterol was purchased from Sigma (Cat. No. C8667). Ethanol and PBS (phosphate buffered saline) were purchased from Sigma. Hyaluronic acid was purchased from Genzyme (Synvisc one, Hylan G-F 20), average molecular weight of hylan A is 6×10⁶ dalton.

### Example 1: Preparation of a CBD/Hyaluronic Acid Liposomal Formulation for Treating Osteoarthritis

250 mg CBD, 787 mg phosphatidylcholine, and 193 mg cholesterol were weighed into a tube and 1 ml ethanol was added. The tube was closed and inserted into a sonication bath pre-heated to 40° C. and sonicated for 10 minutes. The tube was then taken out and vortexed, 1 ml of PBS was added and the tube was inserted into a 50° C. water bath. The contents of the tube were homogenized for 30 minutes using a homogenizer rod for 10 minutes at each of speed levels 3, 4 and 5. The tube was then taken out of the bath and moved to room temperature. Nitrogen gas was used to vaporize the ethanol in the formulation until reaching the desired weight (without ethanol). PBS was added to a final volume of 2.5 ml and the tube was returned to the heated sonication bath for 10 minutes. No more than an hour after the sonication step two syringes was loaded—one with 2.5 ml of hyaluronic acid (total 12 mg, average size 6×10⁶ dalton for hylan A) and the other with 2.5 ml of the formulation, and joined by a male-male Luer-Lock. The formulation was transferred into the syringe containing the hyaluronic acid and back to the original syringe, and this was repeated 10 times until a homogenous solution was obtained. The resulting gel had a white color with lightly foamed consistency. The gel was transferred to a single syringe, which was closed by a Luer-Lock and kept at 4° C.

TABLE 1
ingredients of the formulation

| | | | before | after | |
|---|---|---|---|---|---|
| | | | hyaluronic | hyaluronic | |
| | | | acid | acid | |
| Material | Master formula | | (mg/ml) | (mg/ml) | % W/V |
| CBD | 250 | mg | 100 | 50 | 5 |
| phosphatidylcholine | 787 | mg | 314.8 | 157.4 | 15.7 |
| Cholesterol | 193 | mg | 77.2 | 38.6 | 3.9 |
| Ethanol 96% | 1 | ml | — | | |
| PBS, filtered | 1 | ml | To 2.5 ml | | |

| | | | | |
|---|---|---|---|---|
| Hyaluronic acid | 2.5 ml (12 mg) | — | 2.4 | 0.24 |
| product | | | | |

### Example 2: Effect of the CBD/Hyaluronic Acid Liposomal Formulation on Arthritis in Rats

10 male Wistar rats of about 0.3 kg were anesthetized by 80 mg/kg of Ketamine and 8 mg/ml of Xylazine. Knee osteoarthritis was induced by open medical meniscectomy and medial collateral ligament incision. The disease developed in a time-dependent and predictable fashion. It is a common model assessing the effect of anti-osteoarthritis drugs.

100 microliters of CBD/hyaluronic acid liposomal formulation were injected one week after meniscectomy in one knee of 7 animals The contralateral knee served as control. Hyaluronate (Synvisc One, Genzyme Ltd.) was injected in 3 control animals. The rats were allowed unrestricted motion after the surgery, and evaluated every 1 week using an incapacitance tester, a validated method for assessing pain in rodents following medial meniscectomy. Essentially the technique involves measuring the amount of weight an animal places on an afflicted joint. This test was also used prior to surgery and 24 hours after surgery. The animals were ranked according to the difference between the right and left limbs in terms of weight bearing.

Animal weighing was performed using a standard calibrated animal weighing scale.

As can be seen from FIG. 1, animals injected with the CBD/hyaluronic acid liposomal formulation bore a significantly increase proportion of body weight on the operated knee as compared with control animals that were injected with hyaluronic acid alone (p<0.05), which corresponds to a lessened amount of pain.

### REFERENCES

Ben-Shabat S, Hanus L O, Katzavian G, Gallily R, 2006, New cannabidiol derivatives: synthesis, binding to cannabinoid receptor, and evaluation of their antiinflammatory activity. J. Med Chem. 49 (3):1113-7.

Gui H, et al. 2015, The endocannabinoid system and its therapeutic implications in rheumatoid arthritis. Int Immunopharmacol. 26 (1):86-91.

Haj C G, Sumariwalla P F, Hanus L, Kogan N M, Yektin Z, Mechoulam R, Feldmann M, and Gallily R, 2015, HU-444, a Novel, Potent Anti-Inflammatory, Nonpsychotropic Cannabinoid, J Pharmacol Exp Ther 355:66-75.

Juknat A, Kozela E, Kaushansky N, Mechoulam R, Vogel Z, 2016, Anti-inflammatory effects of the cannabidiol derivative dimethylheptyl-cannabidiol—studies in BV-2 microglia and encephalitogenic T cells. J Basic Clin Physical Pharmacol. 27 (3):289-96.

La Porta C, et al. 2014, Involvement of the endocannabinoid system in osteoarthritis pain. Eur J Neurosci. 39 (3):485-500

Lowin T, et al. 2016, The synthetic cannabinoid WIN55,212-2 mesylate decreases the production of inflammatory mediators in rheumatoid arthritis synovial fibroblasts by activating CB2, TRPV1, TRPA1 and yet unidentified receptor targets. J Inflamm 13:15.

Masuko K, Murata M, Yudoh K, Kato T, Nakamura H, 2009, Anti-inflammatory effects of hyaluronan in arthritis therapy: Not just for viscosity. Int. J. General Medicine 2: 77-81.

McBride, W. H.; Bard, J. B., 1979, J Exp Med. 149 (2), 507-515

Nagarkatti P, Pandey R, Rieder S A, Hegde V L, and Nagarkatti M, 2009, Cannabinoids as novel anti-inflammatory drugs, Future Med Chem. 1 (7): 1333-1349.

Patchornik S, et al. 2012, Chitosan-hyaluronate hybrid gel intraarticular injection delays osteoarthritis progression and reduces pain in a rat meniscectomy model as compared to saline and hyaluronate treatment. Adv Orthop. 2012:979152

Rayahin J E, Buhrman J S, Zhang Y, Koh T J, and Gemeinhart R A, 2015, High and low molecular weight hyaluronic acid differentially influence macrophage activation, ACS Biometer Sci Eng. 1 (7): 481-493.

Sukumar, S.; Drizhal, I., 2007, Acta Medica (Hradec Kralove), 50 (4), 225-228

1. A composition comprising a combination of cannabidiol (CBD) or a derivative thereof, and hyaluronic acid or a salt thereof; a phospholipid, and optionally a carrier.

2. The composition of claim 1, comprising CBD.

3. The composition of claim 1, wherein the hyaluronic acid is a high-molecular-weight hyaluronic acid.

4. The composition of claim 1, wherein the hyaluronic acid is crosslinked.

5. The composition of claim 1, wherein the salt of hyaluronic acid is sodium or potassium hyaluronate.

6. The composition of claim 1, wherein the phospholipid comprises a phosphatidylcholine, a hydrogenated phosphatidylcholine, a lysophosphatidylcholine; dipalmitoylphosphatidylcholine, distearoylphosphatidylcholine, dimyristoylphosphatidylcholine, dilauroylphosphatidylcholine, dioleylphosphatidylcholine, 1-palmitoyl 2-oleyl phosphatidylcholine, a glycerophospholipid, sphingomyelin, cardiolipin, a phosphatidylserine, a phosphatidylglycerol, a phosphatidylinositol, a phosphatidic acid, a phosphatidylcholine-based product, a glycolipid, a plasmalogen, a phosphosphingolipid, Asolectin, lecithin, or a lecithin-like substance, or a mixture thereof.

7. The composition of claim 6, wherein the lecithin is egg lecithin or soybean lecithin; the lecithin-like substance is lecithin egg yolk or soybean oil; or the phosphatidylcholine-based product is Phospholipon® 50, Phospholipon® 75, Phospholipon® 85G, Phospholipon® 90G, Phospholipon® 80H, Phospholipon® 90H, Phospholipon® E25, Phospholipon® E35, Phospholipon® E, Phospholipon® LPC20, Phospholipon® LPC25, or Phospholipon® LPC65.

8. The composition of claim 6, wherein the phospholipid comprises a phosphatidylcholine or a phosphatidylcholine-based product.

9. The composition of claim 8, wherein the phosphatidylcholine-based product is Phospholipon® 90G.

10. The composition of claim 1, further comprising cholesterol.

11. The composition of claim 1, wherein the CBD or derivative thereof is CBD and the hyaluronic acid or a salt thereof is a high molecular weight hyaluronic acid or a salt thereof.

12. The composition of claims 11, wherein the hyaluronic acid or salt thereof is cross-linked.

13. The composition of claim 11, wherein the phospholipid comprises phosphatidylcholine, or a phosphatidylcholine-based product.

14. The composition of claim 13, wherein the phosphatidylcholine-based product comprises Phospholipon® 90G.

15. The composition of claim 13, wherein the weight ratio of CBD/phosphatidylcholine or phosphatidylcholine-based product is between 1/10 and 1/2, preferably 1/3.

16. The composition of any one of claims 1-15, wherein the composition comprises liposomes formed by the phospholipid, and optionally cholesterol, if present, and wherein the CBD or derivative thereof is non-covalently attached to the liposomes.

17. The composition of claim 16, wherein the liposomes are suspended in hyaluronic acid.

18. The composition of claim 16 or 17, comprising CBD; phosphatidylcholine or Phospholipon® 90G; high molecular weight, crosslinked, sodium hyaluronate; and cholesterol, and wherein liposomes are formed from the phosphatidylcholine or Phospholipon® 90G, and the cholesterol; the CBD is non-covalently attached to the liposomes; and the liposomes are suspended in the high molecular weight, crosslinked sodium hyaluronate.

19. The composition of any one of claims 1 to 18, wherein the salt of hyaluronic acid is a pharmaceutically acceptable salt, and the carrier is a pharmaceutically acceptable carrier.

20. A method for treating, or reducing pain or inflammation associated with, an inflammatory joint disease, disorder, or condition in a subject in need thereof, comprising administering to the subject the composition of any one of claims 1-19.

21. The method of claim 20, wherein the inflammatory joint disease, disorder or condition is selected from the group consisting of osteoarthritis, amyloidosis, arthritis bursitis, diffuse idiopathic skeletal hyperostosis (DISH), a ganglion cyst, gout, ankylosing spondylitis, lumbar spinal stenosis, hydroxyapatite juvenile arthritis, pseudogout, SAPHO syndrome, rheumatoid arthritis, reactive arthritis, psoriatic arthritis, sacroiliac joint pain, septic arthritis, Still's disease, and synovitis.

22. The method of claim 21, wherein the inflammatory joint disease, disorder or condition is osteoarthritis.

23. The method of any one of claims 20-22, wherein the administering is by injection.

24. The method of claim 23, wherein the administering is by an intra-articular injection to an inflamed joint.

25. A method for preparing the composition of any one of claims 1-19, wherein the composition comprises cholesterol and the method comprises the following steps:
a) mixing the cannabidiol (CBD), phospholipid and cholesterol to form a mixture;
b) sonicating and homogenizing the mixture; and
c) suspending the mixture in a solution of hyaluronic acid or a salt thereof,
thereby forming liposomes suspended in hyaluronic acid.

26. The method of claim 25, wherein the CBD is between 90% and 99% pure, preferably about 97% pure CBD.

27. The method of claim 25 or 26, wherein the phospholipid is phosphatidylcholine or Phospholipon® 90G, and the weight ratio of CBD/phosphatidylcholine or Phospholipon® 90G is between 1/10 and 1/2, preferably about 1/3.

28. The composition of any one of claims 1-19, for use in treating, or reducing pain or inflammation associated with, an inflammatory joint disease, disorder or condition.
